(19) United States
(12) Patent Application Publication
(10) Pub. No.: US 2019/0029272 A1
(43) Pub. Date: Jan. 31, 2019

(54) Preparation of a Baked Product Comprising Fibers Treated by a Cellulase

(71) Applicant: Novozymes A/S, Bagsvaerd (DK)

(72) Inventor: Helle Niemann, Bagsvaerd (DK)

(73) Assignee: Novozymes A/S, Bagsvaerd (DK)

(21) Appl. No.: 16/072,759

(22) PCT Filed: Feb. 9, 2017
(86) PCT No.: PCT/EP2017/052843

§ 371 Date: Jul. 25, 2018

(30) Foreign Application Priority Data
Feb. 10, 2016 (EP) 16155042.1

(51) Int. Cl. A21D 8/04; C12N 9/42; A21D 13/02

(52) U.S. Cl. CPC A21D 8/042; C12N 9/2437; C12Y 302/01008; C12Y 302/01004; C12Y 302/01091; A21D 13/02

(57) ABSTRACT

The present invention deals with a method of improving properties in a high fiber baked product comprising treating fibers with a cellulase for a period of at least 15 minutes; mixing the cellulase treated fibers with flour and other baking ingredients to make a dough; and baking the dough to produce a baked product.

### FIELD OF THE INVENTION

The present invention relates to a process for preparing a baked product made partly from cereal grain fibers. More particularly, it relates to a process wherein the fibers are pretreated before being incorporated into the dough.

### BACKGROUND OF THE INVENTION

Baked products with high fiber content have become increasingly popular. Foods that are high in fibers are healthy because they make you feel full for longer as the fibers slow the emptying of your stomach.

There is a need for finding improved solutions for high fiber baked products, especially regarding increasing the volume and/or improving the anti-staling properties.

### SUMMARY OF THE INVENTION

The inventor has found that it is possible to improve the properties in a high fiber baked product by having a pretreatment of the fibers, so we claim: A method of improving properties in a high fiber baked product comprising
a) Treating fibers with an enzyme composition comprising a cellulase for a period of at least 15 minutes;
b) Mixing the cellulase treated fibers with flour and other baking ingredients to make a dough; and
c) Baking the dough to produce a baked product.

In one embodiment, the fibers are not heated to 100 degrees Celsius before the cellulase treatment.

In one embodiment, the improved properties are increased volume and/or improved anti-staling properties of the baked product.

In one embodiment, high fiber means that at least 5% (w/w) of the total flour (fiber plus flour) in the dough is fiber.

In one embodiment, the cellulase is obtainable from Trichoderma reesei.

In one embodiment, the cellulase is applied in an amount of 0.01-100 g enzyme protein per kg fiber.

In one embodiment, the treatment of the fibers in step a) is done at a temperature of from 10 degrees Celsius to 60 degrees Celsius.

In one embodiment, additionally a xylanase and/or a cellobiohydrolase is applied in the pretreatment of the fibers (step a).

In one embodiment, additionally an enzyme selected from the group consisting of amylase, alpha-amylase, beta-amylase, maltogenic alpha-amylase, carboxypeptidase, chitinase, cutinase, cyclodextrin glycosyltransferase, esterase, glucanase, galactanase, alpha-galactosidase, beta-galactosidase, glucoamylase, alpha-glucosidase, beta-glucosidase, glucose oxidase, catalase, invertase, lipase, phospholipase, mannosidase, pectinolytic enzymes, peptidoglutaminase, protease, and a phytase, is added in step a) and/or step b).

In one embodiment, the fiber is selected from the group consisting of wheat, barley, rye, oat, corn, sorghum, rice, soy, and millet, and any mixtures thereof.

In one embodiment, the flour is selected from the group consisting of wheat, barley, rye, oat, corn, sorghum, rice, soy, millet, gluten, and any mixtures thereof.

In one embodiment, the baking ingredients are selected from yeast, sugar, salt, water, and oxidants.

In one embodiment, the baked product is selected from the group consisting of loaves, pan bread, toast bread, open bread, pan bread with and without lid, buns, hamburger buns, rolls, baguettes, brown bread, flat bread, tortilla, pita, Arabic bread, Indian flat bread, steamed bread, and any variety thereof.

In one embodiment, the invention claims the use of a cellulase for pretreatment of fibers to be included in dough.

In one embodiment, the baked product obtained by baking the dough according to the invention is claimed.

In one embodiment, the present invention claims improving properties in a high fiber dough comprising providing a mixture of fibers, which have been treated with a cellulase for at least 15 minutes, with flour and other baking ingredients to make a dough.

### DETAILED DESCRIPTION OF THE INVENTION

### Definitions of Improved Property

The term “improved property” is defined herein as any property of dough and/or a product obtained from the dough, particularly a baked product, which is improved by using the method of the present invention.

The improved property may include, but is not limited to, increased strength of the dough, increased elasticity of the dough, increased stability, reduced stickiness of the dough, improved extensibility of the dough, improved machine ability of the dough, increased volume of the baked product, improved flavor of the baked product, improved crumb structure of the baked product, and/or improved crumb softness of the baked product.

Increased strength: The term “increased strength of the dough” is defined herein as the property of dough that has generally more elastic properties and/or requires more work input to mould and shape.

Increased elasticity: The term “increased elasticity of the dough” is defined herein as the property of dough which has a higher tendency to regain its original shape after being subjected to a certain physical strain.

Increased stability of the dough: The term “increased stability of the dough” is defined herein as the property of dough that is less susceptible to mechanical abuse thus better maintaining its shape and volume and is evaluated by the ratio of height:width of a cross section of a loaf after normal and/or extended proof.

Reduced stickiness of the dough: The term “reduced stickiness of the dough” is defined herein as the property of a dough that has less tendency to adhere to surfaces, e.g., in the dough production machinery, and is either evaluated empirically by the skilled test baker or measured by the use of a texture analyzer (e.g., TAXT2) as known in the art.

Improved extensibility: The term “improved extensibility of the dough” is defined herein as the property of dough that can be subjected to increased strain or stretching without rupture.

Improved machine ability: The term “improved machine ability of the dough” is defined herein as the property of a dough that is generally less sticky and/or more firm and/or more elastic.

Increased volume of the baked product: The term “increased volume of the baked product” is measured as the volume of a baked product. The volume may be determined by the rape seed displacement method, or it may be determined as described in the examples.

Improved crumb structure of the baked product: The term “improved crumb structure of the baked product” is defined herein as the property of a baked product with finer cells and/or thinner cell walls in the crumb and/or more uniform/homogenous distribution of cells in the crumb and is usually evaluated visually by the baker or by digital image analysis as known in the art (e. g., C-cell, Calibre Control International Ltd, Appleton, Warrington, UK).

Improved softness of the baked product: The term “improved softness of the baked product” is the opposite of “firmness” and is defined herein as the property of a baked product that is more easily compressed and is evaluated either empirically by the skilled test baker or measured by the use of a texture analyzer (e.g., TAXT2 or TA-XT Plus from Stable Micro Systems Ltd, Surrey, UK) as known in the art.

Improved anti-staling properties of the baked product: The term “improved anti-staling properties of the baked product” is the opposite of “firmness” and is defined herein as the property of a baked product that is more easily compressed and is evaluated either empirically by the skilled test baker or measured by the use of a texture analyzer (e.g. TAXT2 or TA-XT Plus from Stable Micro Systems Ltd, Surrey, UK) as known in the art. The anti-staling properties are typically measured after 1, 2 and/or 3 weeks.

### Enzymes/High Fiber Bread Product

The present invention deals with high fiber baked products made from dough wherein the dough contains enzymatically pretreated fibers.

High fiber baked product: The term “high fiber baked product” is defined as a baked product with whole units, e.g., grains, e.g., whole wheat, and/or are enriched with extra fiber in the form of, e.g., cereal bran, e.g., wheat bran (wheat bran is produced as a side product of milling wheat into white flour).

Normally, fibers are divided into fine fibers, medium fibers, and coarse fibers as known in the art.

Fine fibers are particularly useful in the present invention.

### Cellulases

According to the present invention, the fibers are treated with a cellulase.

The term “cellulase” or “cellulolytic enzyme” as used herein are understood as an enzyme composition or an enzyme mixture comprising a cellulase, in particular an endoglucanase (EC 3.2.1.4).

In one embodiment, the cellulase used in accordance with the present invention is an enzyme composition comprising an endoglucanase (EC 3.2.1.4).

The cellulase may comprise a carbohydrate-binding module (CBM) which enhances the binding of the enzyme to a cellulose-containing fiber and increases the efficacy of the catalytic active part of the enzyme. A CBM is defined as contiguous amino acid sequence within a carbohydrate-active enzyme with a discrete fold having carbohydrate-binding activity. For further information of CBMs see the CAZy internet server or Tomme et al. (1995) in Enzymatic Degradation of Insoluble Polysaccharides (Saddler and Penner, eds.), Cellulose-binding domains: classification and properties, pp. 142-163, American 25 Chemical Society, Washington.

Endoglucanases (E.C. 3.2.1.4) catalyze endo-hydrolysis of 1,4-beta-D-glycosidic linkages in cellulose, cellulose derivatives (such as carboxy methyl cellulose and hydroxy ethyl cellulose), lichenin, beta-1,4 bonds in mixed beta-1,3 glucans such as cereal beta-D-glucans or xyloglucans and other plant material containing cellulosic parts.

Endoglucanase activity may be determined, e.g., by using carboxymethyl cellulose (CMC) hydrolysis according to the procedure of Ghose, 1987, Pure and Appl. Chem. 59: 257-268.

The cellulase mixture may in addition to the endoglucanase include a cellobiohydrolase (E.C. 3.2.1.91) and/or a beta-glucosidase (E.C. 3.2.1.21); in particular a cellobiohydrolase.

A cellobiohydrolase catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain.

Examples of cellobiohydrolases include CBH I and CBH II from Trichoderma reesei.

In some embodiments, the cellulase or the cellulase mixture may be derived from a strain of the genus Trichoderma, such as a strain of Trichoderma reesei; a strain of the genus Humicola, such as a strain of Humicola insolens; or a strain of Chrysosporium, preferably a strain of Chrysosporium lucknowense.

In some embodiments, the enzyme composition for use in the methods and/or uses of the present invention may be the product of expression of one or more enzyme(s) in a suitable host cell (e.g., a fermentation product).

Preferably, the cellulase composition may be obtainable (e.g., obtained) from Trichoderma, preferably from Trichoderma reesei.

An example of a commercial cellulase product produced by Trichoderma reesei is Celluclast BG™, available from Novozymes A/S.

### Xylanases

In one embodiment, the enzyme composition to be used in pretreatment of the fibers, may comprise a xylanase.

The xylanase may preferably be an endo-1,4-beta-xylanase.

The xylanase according to the invention may be of microbial origin, e.g., derived from a bacterium or fungus, such as a strain of Aspergillus, in particular of A. aculeatus, A. niger, A. awamori, or A. tubigensis, or from a strain of Trichoderma, e.g., T. reesei, or from a strain of Humicola, e.g., H. insolens, or from a strain of Meripilus, or from a strain of Fusarium, or from a bacterium (e.g., Bacillus).

Examples of a commercial xylanase include SHEARZYME™ from Novozymes A/S, Denmark.

An enzyme product comprising both a cellulase and a xylanase may also be used, e.g., Ultraflo® Max (available from Novozymes A/S).

### Additional Enzymes

Optionally, an enzyme selected from the group consisting of amylase, alpha-amylase, beta-amylase, maltogenic alpha-amylase, carboxypeptidase, chitinase, cutinase, cyclodextrin glycosyltransferase, esterase, glucanase, galactanase, alpha-galactosidase, beta-galactosidase, glucoamylase, alpha-glucosidase, beta-glucosidase, glucose oxidase, catalase, invertase, lipase, phospholipase, mannosidase, pectinolytic enzymes, peptidoglutaminase, protease, and a phytase, may be added in the pretreatment of the fibers or to the dough.

The additional enzyme may be of any origin, including mammalian and plant, and preferably of microbial (bacterial, yeast or fungal) origin.

The amylase may be fungal or bacterial, e.g., a maltogenic alpha-amylase from B. stearothermophilus or an alpha-amylase from Bacillus, e.g., B. licheniformis or B. amyloliquefaciens, a beta-amylase, e.g., from plant (e.g. soy bean) or from microbial sources (e.g. Bacillus), or a fungal alpha-amylase, e.g., from A. oryzae.

Suitable commercial maltogenic alpha-amylases include NOVAMYL™ and NOVAMYL 3D™ (available from Novozymes A/S).

Suitable commercial fungal alpha-amylase compositions include, e.g., BAKEZYME P 500™ (available from DSM) and FUNGAMYL 2500 SG™, FUNGAMYL 4000 BG™, FUNGAMYL 800 L™, FUNGAMYL ULTRA BG™ and FUNGAMYL ULTRA SG™ (available from Novozymes A/S).

The glucoamylase for use in the present invention includes the A. niger G1 or G2 glucoamylase (Boel et al. (1984), EMBO J. 3 (5), p. 1097-1102), or the A. awamori glucoamylase disclosed in WO 84/02921, or the A. oryzae glucoamylase (Agric. Biol. Chem. (1991), 55 (4), p. 941-949).

Suitable commercial glucoamylases include GoldCrust BG™ (available from Novozymes A/S).

The protease may be from Bacillus, e.g., B. amyloliquefaciens.

The phospholipase may have phospholipase A1, A2, B, C, D or lysophospholipase activity; it may or may not have lipase activity. It may be of animal origin, e.g., from pancreas, snake venom or bee venom, or it may be of microbial origin, e.g., from filamentous fungi, yeast or bacteria, such as Aspergillus or Fusarium, e.g., A. niger, A. oryzae or F. oxysporum. A preferred lipase/phospholipase from Fusarium oxysporum is disclosed in WO 98/26057. Also, the variants described in WO 00/32758 may be used.

Suitable phospholipase compositions are LIPOPAN F™ and LIPOPAN XTRA™ (available from Novozymes A/S) or PANAMORE GOLDEN™ and PANAMORE SPRING™ (available from DSM).

The glucose oxidase may be of any origin, including mammalian and plant, and preferably of microbial (bacterial, yeast or fungal) origin.

The glucose oxidase may be derived from a strain of, e.g., Aspergillus or Penicillium, particularly A. niger, P. notatum, P. amagasakiense or P. vitale.

An example of a commercial glucose oxidase is Gluzyme™, an Aspergillus niger glucose oxidase, available from Novozymes A/S.

A xylanase may also be added to the dough, e.g., a suitable commercially available xylanase preparation for use in the present invention includes PANZEA BG™, PENTOPAN MONO BG™ and PENTOPAN 500 BG™ (available from Novozymes A/S), GRINDAMYL POWERBAKE™ (available from Danisco), and BAKEZYME BXP5000™ and BAKEZYME BXP 5001™ (available from DSM).

### Pretreatment of the Fibers

Enzymatic pretreatments of the fibers are typically done immediately before the fibers are added to the dough.

According to the invention, the fibers may be derived from cereal grain, including wheat, barley, rye, oat, corn, sorghum, rice, soy, and millet, especially wheat. The fibers may also be a mixture of various fibers from different grains.

The fibers are mixed with water. The mixture may be treated with the cellulase at room temperature, or the mixture may be treated with the cellulase a temperature that is optimal for the cellulase to be applied.

The enzymatic treatment of the fibers is typically done at a temperature of from 10° C. to 60° C., e.g., typically at a temperature of from 15° C. to 50° C.; e.g., at a temperature of from 25° C. to 50° C.; e.g., at a temperature of from 35° C. to 50° C.

The cellulase may be applied in an amount of 0.01-100 g enzyme protein per kg fiber, e.g., such as in an amount of 0.1-10 g enzyme protein per kg fiber.

According to the invention, a xylanase may also be added in the pre-treatment of the fibers. The xylanase is typically applied in an amount of 0.01-100 g enzyme protein per kg fiber, e.g., such as in an amount of 0.1-10 g enzyme protein per kg fiber.

The fiber/water/enzyme(s) mixes are incubated at the desired temperature for at least 15 min. The incubation may typically be done within 24 hours, e.g., the incubation may typically be done within 23 hours, e.g., the incubation may typically be done within 22 hours, e.g., the incubation may typically be done within 21 hours, e.g., the incubation may typically be done within 20 hours, e.g., the incubation may typically be done within 19 hours, e.g., the incubation may typically be done within 20 hours, e.g., the incubation may typically be done within 19 hours, e.g., the incubation may typically be done within 18 hours, e.g., the incubation may typically be done within 17 hours, e.g., the incubation may typically be done within 16 hours, e.g., the incubation may typically be done within 15 hours, e.g., the incubation may typically be done within 14 hours, e.g., the incubation may typically be done within 13 hours, e.g., the incubation may typically be done within 12 hours, e.g., the incubation may typically be done within 11 hours, e.g., the incubation may typically be done within 10 hours, e.g., the incubation may typically be done within 9 hours, e.g., the incubation may typically be done within 8 hours, e.g., the incubation may typically be done within 7 hours, e.g., the incubation may typically be done within 6 hours, e.g., the incubation may typically be done within 5 hours, e.g., the incubation may typically be done within 4 hours, e.g., the incubation may typically be done within 3 hours, e.g., the incubation may typically be done within 2 hours, e.g., the incubation may typically be done within 1 hour. The fiber/water/enzyme(s) mixes may be incubated at the desired temperature for ½ h to 12 h; e.g., incubated at the desired temperature for 1 h to 3 h.

After the incubation, the fiber/water/enzyme(s) mixes may be cooled to 25° C. to 30° C., e.g., 30° C., where after the mixes are ready to be used in dough.

It may be an advantage in industrial scale that the fiber/water/enzyme(s) mixes are not tempered/not cooled, whereby energy is saved.

### Dough

The invention discloses a method for preparing high fiber dough or a high fiber baked product prepared from the dough which method comprises incorporating into the dough enzymatically treated fibers.

According to the present invention, high fiber means that at least 5% (w/w) of the total flour (flour plus fiber) in the dough is fiber, e.g., at least 10% (w/w) of the total flour (flour plus fiber) in the dough is fiber, e.g., at least 15% (w/w) of the total flour (flour plus fiber) in the dough is fiber, e.g., at least 20% (w/w) of the total flour (flour plus fiber) in the dough is fiber, e.g., at least 25% (w/w) of the total flour (flour plus fiber) in the dough is fiber, e.g., at least 30% (w/w) of the total flour (flour plus fiber) in the dough is fiber, e.g., at least 35% (w/w) of the total flour (flour plus fiber) in the dough is fiber, e.g., at least 40% (w/w) of the total flour (flour plus fiber) in the dough is fiber, e.g., at least 45% (w/w) of the total flour (flour plus fiber) in the dough is fiber, e.g., at least 50% (w/w) of the total flour (flour plus fiber) in the dough is fiber, e.g., at least 55% (w/w) of the total flour (flour plus fiber) in the dough is fiber, e.g., at least 60% (w/w) of the total flour (flour plus fiber) in the dough is fiber, e.g., at least 65% (w/w) of the total flour (flour plus fiber) in the dough is fiber, e.g., at least 70% (w/w) of the total flour (flour plus fiber) in the dough is fiber, e.g., at least 75% (w/w) of the total flour (flour plus fiber) in the dough is fiber, e.g., at least 80% (w/w) of the total flour (flour plus fiber) in the dough is fiber, e.g., at least 85% (w/w) of the total flour (flour plus fiber) in the dough is fiber, e.g., at least 90% (w/w) of the total flour (flour plus fiber) in the dough is fiber, e.g., at least 95% (w/w) of the total flour (flour plus fiber) in the dough is fiber.

The fiber content in the dough will typically be from 5% (w/w) to 90% (w/w) of the total flour (flour plus fiber).

The term “dough” is defined herein as a mixture of flour and other baking ingredients firm enough to knead or roll.

The dough of the invention may comprise flour derived from grain, wheat, barley, rye, oat, corn, sorghum, rice, soy, millet, and gluten, especially wheat, or any mixtures thereof.

The dough may also comprise other conventional dough ingredients, e.g., such as milk, milk powder, and eggs (whole eggs, egg yolks, and/or egg whites).

The dough may also comprise one or more oxidants such as ascorbic acid, potassium bromate, potassium iodate, azodicarbonamide (ADA), or ammonium persulfate.

The dough may also comprise an amino acid such as L-cysteine; a sugar (e.g., sucrose); a salt such as sodium chloride, calcium acetate, sodium sulfate, or calcium sulfate.

The dough may also comprise fat (triglyceride) such as butter, margarine, granulated fat or shortening.

The dough may also comprise an emulsifier selected from the group consisting of diacetyl tartaric acid esters of monoglycerides (DATEM), sodium stearoyl lactylate (SSL), calcium stearoyl lactylate (CSL), ethoxylated mono- and diglycerides (EMG), polysorbates (PS), succinylated monoglycerides (SMG), distilled monoglycerides (DMG), and mono- and diglycerides (MDG), and mixtures thereof.

The dough of the invention may be fresh, frozen or par-baked (pre-baked).

The dough of the invention is normally leavened dough or dough to be subjected to leavening. The dough may be leavened in various ways, such as by adding chemical leavening agents, e.g., sodium bicarbonate or by adding a leaven (fermenting dough), but it is preferred to leaven the dough by adding a suitable yeast culture, such as a culture of Saccharomyces cerevisiae (baker's yeast), e.g., a commercially available strain of S. cerevisiae.

### Baked Product

The process of the invention may be used for any kind of baked product prepared from high fiber dough, either of a soft or a crisp character.

Examples of baked products are bread typically in the form of loaves or rolls, pan bread, toast bread, pan bread with and without lid, buns, hamburger buns, rolls, baguettes, brown bread, whole meal bread, rich bread, bran bread, flat bread, tortilla, pita, Arabic bread, Indian flat bread, steamed bread, and any variety thereof.

The present invention is further described by the following example that should not be construed as limiting the scope of the invention.

### EXAMPLES

### Example 1

Baking Trials with Enzymatically Pretreated Fiber

The overall process for the baking trials was the following:
A. Enzymatic fiber pretreatment.
B. Determination of water absorption of dough made with 20% enzymatically pretreated fiber using Mixolab.
C. Baking trial with 20% enzymatic pretreated fiber.
D. Volume determination of bread baked with 20% enzymatic pretreated fiber.
E. Determination of anti-staling properties of bread baked with 20% enzymatically pretreated fiber.

### A. Enzymatic Fiber Pretreatment

Enzymatic pretreatments of fibers were done immediately before use in either the determination of optimal content or the baking trial.

Three different types of treatment were used (see Table 1).

For each treatment, 27 ml of tap water was tempered at 45° C. (±1° C.) and mixed with enzymes according to Table 1 in a glass beaker.

9 g of wheat bran (Tarwezenmelen fijn, Meneba, The Netherlands) were mixed with each enzyme solution. The fiber/water mixes were incubated at 45° C. for 2 h. The fiber/water mixes were cooled to 30° C. by placing the beaker in a water bath with ice. The enzymatically pretreated fibers were used immediately.

TABLE 1
Composition of enzymatic pretreatment of fiber samples

| | Name | | |
|---|---|---|---|
| | | | 2% Ultraflo |
| | Reference | 2% Celluclast | Max |
| Fine fiber (g) | 9 | 9 | 9 |
| Water (g) | 27 | 27 | 27 |
| Celluclast BG (g) | 0 | 0.18 | 0 |
| Ultraflo Max (g) | 0 | 0 | 0.18 |

B. Determination of Water Absorption of Dough Made with Enzymatically Pretreated Fibers Using Mixolab

The water absorption of dough made from 20% (w/w) enzymatically pretreated fiber and 80% (w/w) wheat flour (Pelikaan, Meneba, The Netherlands) was determined according to the Mixolab simulator (Chopin S, Mixolab, France).

The water absorption for each dough was used in subsequent baking trials in order to produce dough with the same and optimal consistency.

### Procedure:

1. 36 g of wheat flour (Pelikaan, Meneba, The Netherlands) was added to the Mixolab mixing chamber.
2. 36 g of enzymatically pretreated fiber slurry (containing 9 g fine wheat fiber and 27 g water) was added to the mixolab mixing chamber.
3. The mixolab was started and run according to the Chopin S protocol. Specifically, trials were run at 30° C. with a mixing speed of 80 rpm for 12 min, the resistance to mixing the dough (torque, Nm) was recorded as function of time (s).
4. The volume of added water was varied to reach the maximum torque of 1.1+/−0.05 Nm (C1).
5. The water absorption of the dough containing the enzymatically treated fibers was calculated as the sum of water added with the enzyme treated fiber slurry and the water added to the mixolab (Table 2.).

TABLE 2
Determination of water absorption of dough with enzymatically
pretreated fibers.

| | Dough number | | |
|---|---|---|---|
| | 1 | 2 | 3 |
| Name | Control | 2% Celluclast | 2% Ultraflo |
| | | | Max |
| Pelikaan flour (Meneba, The | 36 | 36 | 36 |
| Netherlands) (g) | | | |
| Fiber slurry from fiber pre- | 36 | 36 | 36 |
| treatment (g) (comprising of 9 g | | | |
| fiber and 27 g water) | | | |
| Added water to mixolab to | 4.9 | 1.2 | 0.74 |
| reach 1.1 Nm (g) | | | |
| Sum of added water (g) | 31.9 | 28.2 | 27.74 |
| Water absorption (%) | 71 | 63 | 62 |

C. Baking Trial with Enzymatically Pretreated Fibers

The baking procedure was run as a two steps process where the first step was an enzymatic fiber pretreatment and the second step was a normal straight dough baking trial where the bread was baked in open pans. Four baking trials were performed.

### Process:

1. Enzymatic fiber pretreatment was performed as described in section A, however, the levels of water were adjusted to the water adsorption percent shown in Table 2. The enzymatic pretreatment of fibers for baking trials was performed with 500 g of fibers as starting material, all other components were scaled up accordingly (Table 3). For the 2^{nd} sample (Benchmark) the fiber pretreatment was performed as for sample 1 (Control) with the addition that 7.5 g DATEM was added as an additional ingredient along with the other ingredients.
2. The enzymatic pretreated fibers were added to the mixing bowl of a pin mixer (Bjorn mixer RN20NL2, Denmark) along with all the other ingredients according to the recipe of the baking trials (Table 4).
3. The ingredients were mixed for 2 min at 50 rpm and then for 6 min at 150 rpm into a dough.
4. The dough was allowed to rest on the bench covered with plastic film for 10 min.
5. The dough was scaled to 500 g pieces and rounded in a rounder (Werner & Pfleiderer, CR59, Panningen, The Netherlands), and were placed in a climate chamber for 40 min at 86% rh, 29° C.
6. The dough was sheeted (MO-671, Glimek, Sweden), placed in a pan and proofed in a proofing cabinet at 86% rh, 29° C. for 75 min.
7. The dough was baked in a deck oven for 30 min at 225° C.
8. The bread was allowed to cool down to 30° C. for 2 h at room temperature.
9. The volume of the bread was determined in Volscan Profiler, Stable microsystems, Godalming, UK.
10. The bread was placed in sealed plastic bags with modified atmosphere (100% N₂) and stored at room temperature.
11. The texture of the bread was determined on day 2 and day 7 using TA-XTplus texture analyzer, Stable microsystems, Godalming, UK.

TABLE 3
Enzymatic pre-treatment of wheat bran for baking trial

| | Sample number | | | |
|---|---|---|---|---|
| | 1 | 2 | 3 | 4 |
| Name | Control | Benchmark | 2% Celluclast | 2% Ultraflo |
| | | (0.3% | BG | Max |
| | | DATEM) | | |
| Fine wheat bran, | 500 | 500 | 500 | 500 |
| Meneba, NL (g) | | | | |
| Water (g) | 1767.5 | 1767.5 | 1567.5 | 1542.5 |
| Celluclast BG (g) | 0 | 0 | 10 g | 0 |
| Ultraflo Max (g) | 0 | 0 | 0 | 10 g |

TABLE 4
Recipe for straight dough European style bread

| Ingredient | Amount |
|---|---|
| Flour Pelikaan, Menba | 80% |
| Enzymatically pretreated | 20% |
| fiber | |
| Water | Vary according to water absorption |
| | determined in mixolab (All the water was |
| | added in the fiber pretreatment step) |
| Yeast | 3% |
| Sucrose | 1% |
| Salt | 2% |
| Ascorbic acid | 60 ppm |
| Calcium propionate | 0.25% |

D. Volume Determination of the Bread Baked with 20% Enzymatically Pretreated Fiber

The volume of the breads with enzymatically pretreated fibers was determined in a Volscan profiler (Stable microsystem, Godalming, UK).

### Procedure:

1. The bread was mounted and fixed in a vertical position in the instrument by placing one end of the bread on a sample holder so that the three needles pierce into the bread loaf. On the opposite end of the bread, a pin via the upper supporting arm, was inserted into the bread.
2. The weight of the bread was recorded automatically in grams by the built-in balance of the Volscan profiler instrument.
3. A three dimensional shape of the bread was created by scanning the contour of the bread with a laser moving from the bottom of the bread to the top while the bread was rotating at a speed of 90 rpm.
4. From the generated three dimensional shape, the volume of the bread was calculated.
5. The specific volume of the bread (in ml/g) was calculated by dividing the volume of the bread in ml by the weight of the bread in grams as can be found in Table 5.

TABLE 5
Specific volume ml/g

| | | Specific vol. |
|---|---|---|
| Treatment | Name | (mL/g) |
| 1 | Control | 2.74 |
| 2 | DATEM 0.3% | 3.56 |
| 3 | Celluclast BG, 2% | 3.75 |
| 4 | Ultraflo Max, 2% | 4.02 |

### Conclusion

The fiber, pretreated with Celluclast or Ultraflo Max, produced bread with larger volume than bread produced without addition of enzyme (Control).

A common method to increase the volume of the baked product is to add the emulsifier DATEM at a level of 0.3%. The addition of DATEM to bread baked with pretreated fibers without addition of enzyme increased the volume, but not to the same degree as fiber pretreated with Celluclast BG or Ultraflo Max.

E. Determination of Anti-Staling Properties of Bread Baked with 20% Enzymatically Pretreated Fiber

The change in hardness of the bread was determined with a TA-XT plus texture analyzer, (Stable Micro Systems Ltd, Godalming, UK)

### Process

1. The bread was stored in sealed plastic bags until analysis at room temperature, the bread was analyzed on day 2 and 7 after baking.
2. The bread was sliced in 20 mm thick slices. The three slices from the center of the bread were used for texture analysis. For each treatment and day, two replicas were analyzed.
3. The bread slice was placed on a texture analyzer so that the probe will press down in the center of the slice. A cylindrical probe with a diameter of 45 mm was pressed down into the center of the bread slice to a 40% strain. The probe was held in this position for 33 s, and then the probe returned to its original position.
4. The hardness of the bread crumb was calculated as the force required to press the probe with a 30% strain into the bread.
5. The hardness summarized in Table 6 is an average of six measurements (6 slices).

TABLE 6
Hardness of bread slices on day two and seven after baking

| | | day two | day seven |
|---|---|---|---|
| Treatment | Name | (g) | (g) |
| 1 | Control | 1301 | 2138 |
| 2 | DATEM, 0.3% | 662 | 1270 |
| 3 | Celluclast BG | 981 | 1528 |
| 4 | Ultraflo Max | 905 | 1644 |

### Conclusion

The fiber pretreated with Celluclast BG or Ultraflo Max produced breads that were softer than bread with pretreated fiber but without addition of enzyme (treatment 1, control).

The increase in hardness between day 2 and 7 was lower for the bread baked with enzymatically pretreated fibers indicating that the pretreatment of the fiber reduced the staling rate.

1. A method of improving properties in a high fiber baked product comprising
a. Treating fibers with an enzyme composition comprising a cellulase for a period of at least 15 minutes;
b. Mixing the cellulase treated fibers with flour and other baking ingredients to make a dough; and
c. Baking the dough to produce a baked product.

2. The method according to claim 1, wherein the fibers are not heated to 100 degrees Celsius before the cellulase treatment.

3. The method according to claim 1, wherein the improved properties are increased volume and/or improved anti-staling properties of the baked product.

4. The method according to claim 1, wherein high fiber means that at least 5% (w/w) of the total flour (fiber plus flour) in the dough is fiber.

5. The method according to claim 1, wherein the cellulase is obtainable from Trichoderma reesei.

6. The method according to claim 1, wherein the enzyme composition comprising a cellulase is applied in an amount of 0.01-100 g enzyme protein per kg fiber.

7. The method according to claim 1, wherein the treatment of the fibers in step a) is done at a temperature of from 10 degrees Celsius to 60 degrees Celsius.

8. The method according to claim 1, wherein additionally a xylanase and/or a cellobiohydrolase is applied in step a).

9. The method according to claim 1, wherein additionally an enzyme selected from the group consisting of amylase, alpha-amylase, beta-amylase, maltogenic alpha-amylase, carboxypeptidase, chitinase, cutinase, cyclodextrin glycosyltransferase, esterase, glucanase, galactanase, alpha-galactosidase, beta-galactosidase, glucoamylase, alpha-glucosidase, beta-glucosidase, glucose oxidase, catalase, invertase, lipase, phospholipase, mannosidase, pectinolytic enzymes, peptidoglutaminase, protease, and a phytase, is added in step a) and/or step b).

10. The method according to claim 1, wherein the fiber is selected from the group consisting of wheat, barley, rye, oat, corn, sorghum, rice, soy, and millet.

11. The method according to claim 1, wherein the flour is selected from the group consisting of wheat, barley, rye, oat, corn, sorghum, rice, soy, millet, and gluten.

12. The method according to claim 1, wherein baking ingredients are selected from yeast, sugar, salt, water, and oxidants.

13. The method according to claim 1, wherein a baked product is selected from the group consisting of loaves, pan bread, toast bread, open bread, pan bread with and without lid, buns, hamburger buns, rolls, baguettes, brown bread, flat bread, tortilla, pita, Arabic bread, Indian flat bread, steamed bread, and any variety thereof.

14. (canceled)

15. (canceled)

16. A method of improving properties in a high fiber dough comprising providing a mixture of fibers, which have been treated with a cellulase for at least 15 minutes, with flour and other baking ingredients to make a dough.
